(19) United States
(12) Patent Application Publication
(10) Pub. No.: US 2006/0173343 A1
(43) Pub. Date: Aug. 3, 2006

(54) Grounded interleaved flex for ultrasound transducer array

(75) Inventors: Xiaocong Guo, Woodinville, WA (US); Grazyna M. Palczewska, Bellevue, WA (US); Todor Sheljaskow, Issaquah, WA (US); Ellen M. Rowland, Bellevue, WA (US); Haifeng Wang, Issaquah, WA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/015,085

(22) Filed: Dec. 17, 2004

(51) Int. Cl. A61B 8/14

(52) U.S. Cl. 600/459

(57) ABSTRACT

Electronic cross-talk is reduced in an ultrasound transducer. Ground traces are interleaved with signal traces on a flexible film. By providing ground traces between the signal traces, the electrical cross-talk between signal traces is reduced.

### BACKGROUND

The present invention relates to flexible circuit material connectors for ultrasound transducer arrays. In particular, trace patterns are provided for ultrasound transducer arrays.

Ultrasound transducers include an array of elements, such as a one-dimensional or multi-dimensional array of elements. The elements are connected to transmit or receive circuitry of an ultrasound imaging system. Adjacent to the transducer, the electrical connections are provided by flexible circuit material with associated traces. An electrode is provided for each of the elements of the array on the flexible material. A via then connects a signal trace through the flexible material to the electrode. The signal traces from the various elements of the array are routed on a back side of the flexible film for connection to coaxial cables or circuitry.

For a one-dimensional transducer array, the elements have sufficient separation to allow for minimum electrical cross-talk between signal traces on the flexible film. For multi-dimensional transducer arrays, the traces are more densely provided on the flexible film, resulting in an increased electrical cross-talk. As a result of electrical cross-talk, even for one-dimensional arrays, an acceptance angle may be reduced.

### BRIEF SUMMARY

By way of introduction, the preferred embodiments described below include methods and systems for reducing electronic cross-talk in an ultrasound transducer. Ground traces are interleaved with signal traces on a flexible film. By providing ground traces between the signal traces, electrical cross-talk between signal traces is reduced.

In a first aspect, a system is provided for reducing electronic cross-talk in an ultrasound transducer. A plurality of signal traces are provided on flex material. The signal traces connect with respective elements of an ultrasound transducer array. A ground trace is also provided on the flex material. The ground trace is positioned between two of the signal traces.

In a second aspect, a system is provided for reducing electronic cross-talk in an ultrasound transducer. A multi-dimensional array has ultrasound transducer elements. A flexible film has signal traces separated by ground traces. The signal traces connect with the ultrasound transducer elements.

In a third aspect, a method is provided for reducing electronic cross-talk in an ultrasound transducer. Electrical signals are passed to or from ultrasound transducer elements along first paths of a flexible film. Second paths on the flexible film are grounded during the passing of the electrical signals on the first path. The second paths are interleaved with the first path.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a top view of one embodiment of a system for reducing electrical cross-talk;

FIG. 2 is a cross-sectional diagram of the embodiment of FIG. 1;

FIG. 3 is a partial or sectional view of traces on a flexible film in one embodiment;

FIG. 4 is a cross-sectional view perpendicular to signal traces in one embodiment; and

FIG. 5 is a flow chart diagram of one embodiment of a method for reducing electrical cross-talk.

### DETAILED DESCRIPTION OF THE DRAWINGS AND PRESENTLY PREFERRED EMBODIMENTS

A flex with interleaved traces is provided for use with transducer arrays. For example, an interleaved double sided flex is provided for use with two-dimensional transducer arrays. Ground traces are provided between signal traces to decrease inter-trace capacitance. Reduction in inter-trace capacitance reduces electronic cross-talk and may result in an increased acceptance angle.

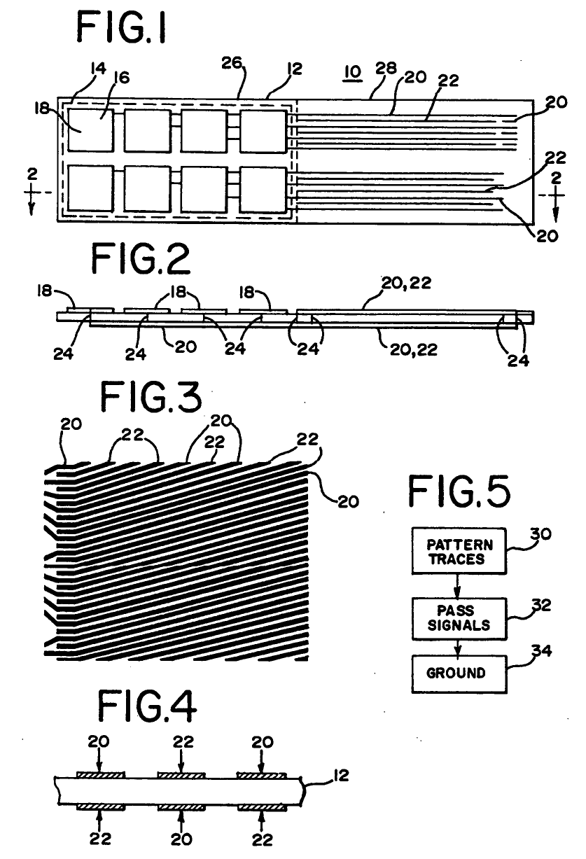

FIGS. 1 and 2 show one embodiment of a system 10 for reducing electrical cross-talk in an ultrasound transducer. The components of the system 10 are provided in the transducer housing, such as within a hand-held, intraoperative, catheter, endocavity or other medical diagnostic ultrasound transducer. The system 10 includes an ultrasound transducer array 14 of elements 16, and flex material 12. Additional, different or fewer components may be provided, such as providing a connector on the flex material 12, a housing, electronic circuitry, co-axial cables or other now known or later developed ultrasound transducer components.

The ultrasound transducer array 14 is a one-dimensional, multi-dimensional or two-dimensional array of elements 16. Multi-dimensional arrays of elements include 1.25, 1.5, 1.75, or 2D transducer arrays. A two-dimensional array includes both square and rectangular distributions of elements. A fully or sparsely sampled element distribution may be provided. Rectangular, triangular, hex or other distribution grids of the elements 16 are used. While eight elements 16 are shown in FIG. 1 for simplicity, additional or fewer elements may be provided. For example, one embodiment of a multi- or two-dimensional array 14 includes 1,728 elements in a rectangular fully sampled grid of 48 elements 16 in azimuth by 36 elements 16 in an elevation direction. The pitch size is 400 micrometers. The pitch size is equal in both azimuth and elevation directions, but may be unequal in other embodiments. Other number of elements 16, different distributions, and/or different pitch sizes may be provided.

The flex material 12 is a flexible film with signal traces 20 separated by ground traces 22. Any of various materials may be used, such as polyimide, polyester, nylons, composites or other flexible circuit materials. In one embodiment, the flex material 12 is Kapton from Dupont Company. The flex material 12 is 50 micrometers thick, but other thicknesses may be provided.

The flexible material 12 is sized to fit under the foot-print of the transducer array 14 and provide a section 28 for connection with other components such as an electrical circuit. As shown in FIG. 1, the foot-print section 26 is slightly larger than the transducer array 14 or a section of the transducer array 14, but may be larger or smaller. The circuit connection section 28 of the flex material 12 extends in one direction away from the foot-print section 26, but circuit connection sections 28 extending in opposite directions, adjacent directions or at other angles relative to the transducer array 14 may be provided.

The signal traces 20 and ground traces 22 are provided on the flex material 12. The traces are copper (e.g. rolled annealed copper), gold or other now known or later developed conductors. The traces 20, 22 are patterned by deposition, etching, doping, bonding, or other now known or later developed patterning techniques for placing traces 20, 22 on flex material 12. In one embodiment for use at a center frequency of about 2.5 MHz, each of the traces 20, 22 is an elongated line about 25 microns in width. About 25 microns separates each trace 20, 22 from other traces 20, 22. Greater or lesser trace widths or separations may be provided for use at the same or different frequencies.

The signal traces 22 connect with respective ones of the elements 16 of the transducer array 14. For example, the signal traces 20 extend from the circuit connection section 28 into the transducer array foot-print section 26 of the flexible material 12. Using vias, deposited insulator layers or other electrical isolation, the signal traces 20 are separately routed to respective ones of the transducer elements 16. Electrodes 18 are formed on the flex material 12 for connection with individual elements 16. An electrode plane may be separated by the same dicing used to form elements 16 or the electrodes 18 are separately deposited or formed on the flex material 12. Each electrode 18 is associated with a signal trace 20 and element 16. The signal traces 20 are operable to transmit the electrical signals to or from the elements 16 and associated circuitry. In alternative embodiments, a given signal trace 20 may connect with two or more electrodes 18 or elements 16.

The ground traces 22 are positioned between the signal traces 20. For example, a given ground trace 22 is positioned between two adjacent signal traces 20. The ground traces 22 may be interconnected to form a single trace on the same flex material 12 or may be commonly connected to ground through a connector. The ground traces 22 are interleaved with the signal traces 20. For example, every other trace along a plurality of parallel traces is a ground trace 22 and the other traces are signal traces 20. FIG. 3 shows one embodiment of the trace pattern with generally parallel ground traces 22 separating adjacent signal traces 20. FIG. 3 is a cutaway portion of a section of the flex material 12, such as the circuit connection section 28 and an interface with the transducer foot-print section 26. In one embodiment, the ground traces 22 are provided as the outer most traces on the flex material 12, but signal traces 20 may be provided as the outer most traces.

Referring to FIGS. 1, 2 and 3, the ground traces 22 extend along a portion of the flex material 12 with the signal traces 20, such as providing the ground traces 22 only in the circuit connection section 28 of the flex material 12. By providing ground traces shorter than the signal traces, the signal traces 20 may be more densely packed in one section free of one or more ground traces 22. By providing ground traces 22 along at least a portion of the signal traces 20, the electrical cross-talk between adjacent signal traces may be reduced. In alternative embodiments, the ground traces 22 extend substantially along the entire length of the signal traces 20.

A single sided flex material 12 is provided in one embodiment. The signal and/or ground traces 22 are provided on a single side of the flex material 12, such as a bottom side. The electrodes 18 for connection with the transducer elements 16 are positioned on a top side. Vias 24 connect the electrodes 18 with signal traces 20 on the bottom side. Ground traces 22 are provided on the bottom side for regions of the flex material 12 spaced away from the foot-print of the transducer array 14. Alternatively, the ground traces 22 extend on the bottom side to portions of the flex material 12 associated with the foot-print of the array 14.

Alternatively, the flex material 12 is used as a double sided flexible circuit. The signal traces 20 and/or ground traces 22 are provided on both the top and bottom sides of the flex material 12. For the transducer foot-print section 26, the signal traces 20 are provided on a bottom side with vias 24 connecting the signal traces 22 to the electrodes 18. Using vias 24, some, such as half, of the signal traces 20 are routed to a top or opposite side of the flex material 12 in the circuit connection section 28. On the circuit connection section 28, the double sided use of the signal traces 20 results in half the density of the signal traces 20 as compared with the foot-print section 26. Given the lesser density, ground traces 22 are interleaved on both the top and bottom opposite sides of the flex material 12 in the circuit connection section 28. A connector is provided for connecting the signal traces 20 on both sides of the flexible material to the circuitry. Alternatively, additional vias 24 are provided for routing the signal traces 20 from the bottom or top sides to the other side, resulting in all or most of the signal traces 20 on a single side of the flex material 12 immediately adjacent to or under a connector. The ground traces 22 are shorter to allow for the increased density due to the connector routing of the signal traces 20. For example, vias 24 are provided to route the ground traces 22 of both sides to a common ground connection.

FIG. 4 shows one embodiment of a dual sided use of the flex material 12. The signal traces 20 and ground traces 22 are interleaved on both sides. The signal traces 20 of one side are opposite ground traces 22 on the other side. By aligning the ground traces 22 beneath or over signal traces 20 on an opposite side, the signal traces 20 are more greatly electrically isolated from other signal traces 20. In alternative embodiments, the pattern of traces 20, 22 on a top surface is independent of a pattern provided on the bottom surface. Other relationships of traces 20, 22 of the top versus bottom surface may be provided.

In one embodiment, a single sheet of flex material 12 and associated traces 20, 22 are provided for an entire multi-dimensional array. The transmit and receive operations are performed over the same signal traces 20. In alternative embodiments, two or more sheets of flexible material 12 and associated traces 20, 22 are provided for the same array. For example, one sheet of flex material 12 is provided on a top surface of ultrasound transducer element 16 for transmit or receive operation, and another sheet of flexible material 12 is provided on a bottom surface of the transducer elements 16 for the other of receive or transmit operation. Additionally or alternatively, a multi-dimensional array 14 is divided into sections. For example, a two-dimensional array 14 includes 1,728 elements at a 400 micrometer pitch with 48 elements in the azimuth direction and 36 elements in the elevation direction. A flexible material 12 with traces 20, 22 is provided for each group of 576 elements in a 12 elevation by 46 azimuth arrangement. The signal traces 20 of different sheets of flex material 12 connect with different groups of elements 16. Other array sizes, pitches, or divisions for flexible material 12 arrangements may be provided.

FIGS. 1 and 3 show signal and ground traces 20, 22 extending in a single axis aligned with or at an angle from a transducer array axis, such as the azimuth or elevational axis. In alternative embodiments, some of the traces 20, 22 extend in different directions, such as in opposite direction from the transducer array 14 or at other angles relative to the transducer array. In yet other embodiments, the signal and ground traces 20 and 22 are not parallel, such as extending in a fan or with traces diverging from each other away from the transducer array 14. The ground traces 22 may expand or vary in width as a function of the divergence of the signal traces 20. In one embodiment, the signal traces 20 extend generally or at a small angle along an elevation axis.

Any amount of reduction of cross-talk may be provided, such as reduction of about 50 percent. In alternative embodiments, no reduction of cross-talk is provided. Similarly, the change in acceptance angle may be improved or remain the same. For example, the traces pattern as shown in FIG. 3 may provide an increase in acceptance angle of 7 to 9 degrees as compared to a pattern without the grounded traces 22. The cross-talk may be reduced by 6 to 8 dB, but greater or lesser reductions may be provided.

FIG. 5 shows one embodiment of a method for reducing electronic cross-talk in an ultrasound transducer. The method is implemented using the system 10 shown in FIG. 1 or other systems. Additional, different or fewer acts may be provided.

In act 30, traces are patterned on one or more flexible films. Patterning is performed by etching, deposition, doping or other now known or later developed techniques for forming traces on a flexible film. The patterning allows for isolation of signal traces from each other. For example, ground or other DC voltage traces are provided between the signal traces. In yet another example, signal traces associated with no use at one time are positioned between signal traces associated with use at that time period.

In act 32, electrical signals are passed to or from ultrasound transducer elements. The electrical signals are passed in transmit or receive operations along paths on a flexible film. The paths are formed from signal traces. The electrical signals correspond to a waveform for transmission by a transducer or electrical signal received in response to acoustic echoes. The electrical signals are passed along traces on one or two sides of the flexible film.

In act 34, other paths on the flexible film are grounded while the electrical signals are passed in act 32. The other paths are interleaved with the paths for carrying the electrical signals. For example, every other path is grounded. In alternative embodiments, every third, every fourth or other frequencies of interleaving are provided. The grounding is provided with a permanent connection, such as without any switching, in one embodiment. In other embodiments, a switch or other selection device is used for selectively grounding particular paths. Different paths may be grounded at different times. Where a two sided flexible film is used, the paths on both sides of the flexible film are grounded. Alternatively, paths on one side are grounded and paths on an opposite side are free of grounding connection.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for reducing electronic cross-talk in an ultrasound transducer, the system comprising:
an ultrasound transducer array of elements;
flex material;
a plurality of signal traces on the flex material, the signal traces connected with respective ones of the elements of the ultrasound transducer array; and
a ground trace on the flex material, the ground trace positioned between two of the signal traces.

2. The system of claim 1 wherein the ultrasound transducer array of elements comprises a multi-dimensional array of elements.

3. The system of claim 2 wherein the ultrasound transducer array of elements comprises a two-dimensional array of elements.

4. The system of claim 1 wherein the flex material comprises a polymide film.

5. The system of claim 1 wherein the flex material comprises a first sheet of material with the signal traces connected with a first set of the elements;
further comprising:
a separate, second sheet of flexible material having signal traces connected with a second set of elements.

6. The system of claim 1 wherein the ground trace comprises a plurality of ground traces, the ground traces and signal traces interleaved on the flex material.

7. The system of claim 6 wherein the flex material comprises a transducer array foot-print section and a circuit connection section, wherein the signal traces extend within both the transducer foot-print section and the circuit connection section, and wherein ground traces terminate within the circuit connection section, the transducer array foot-print section free of the ground traces.

8. The system of claim 7 wherein the plurality of signal traces comprises signal traces connected with respective electrodes with first vias, the electrodes positioned adjacent to the elements on a first side in the transducer foot-print section, and the signal traces in the transducer section on a second side of the flex material opposite the first side, about half of the signal traces extending through second vias to the first side in the circuit connection section, the ground traces on the first and second sides of the flex material in the circuit connection section.

9. The system of claim 6 wherein a first set of interleaved signal traces and ground traces are on a first side of the flex material and a second set of interleaved signal traces and ground traces are on an opposite, second side of the flex material.

10. The system of claim 9 wherein the signal traces of the first set are opposite ground traces of the second set and ground traces of the first set are opposite signal traces of the second set.

11. The system of claim 6 wherein the ground traces separate adjacent signal traces along at least a portion of the flex material.

12. A system for reducing electronic cross-talk in an ultrasound transducer, the system comprising:
a multi-dimensional array of ultrasound transducer elements; and
a flexible film having signal traces separated by ground traces, the signal traces connected with the ultrasound transducer elements.

13. The system of claim 12 wherein the flexible film and at least an additional flexible film have signal traces connecting with the multi-dimensional array.

14. The system of claim 12 wherein the flex material comprises a transducer array foot-print section and a circuit connection section, wherein the signal traces extend within both the transducer foot-print section and the circuit connection section, and wherein ground traces terminate within the circuit connection section, the transducer array foot-print section free of the ground traces.

15. The system of claim 12 wherein a first set of signal traces and ground traces are on a first side of the flexible film and a second set of signal traces and ground traces are on an opposite, second side of the flexible film.

16. The system of claim 15 wherein the signal traces of the first set are opposite ground traces of the second set and ground traces of the first set are opposite signal traces of the second set.

17. The system of claim 12 wherein the signal and ground traces comprise elongated electrical conductors on the flexible film, every other trace being a ground trace separating adjacent signal traces.

18. A method for reducing electronic cross-talk in an ultrasound transducer, the method comprising:
(a) passing electrical signals to or from ultrasound transducer elements along first paths on a flexible film; and
(b) grounding second paths on the flexible film during (a), the second paths interleaved with the first paths.

19. The method of claim 18 wherein (b) comprises grounding the second paths with a permanent connection.

20. The method of claim 18 wherein (a) comprises passing along the first paths on first and second sides of the flexible film, and wherein (b) comprises grounding the second paths of the first and second sides of the flexible film.
